(19) United States
(12) Patent Application Publication
(10) Pub. No.: US 2007/0092559 A1
(43) Pub. Date: Apr. 26, 2007

(54) Liquid dosage forms having enteric properties of delayed and then sustained release

(76) Inventors: Jinghua Yuan, Kingsport, TN (US); Nancy Meade Clipse, Mount Carmel, TN (US)

Correspondence Address: Eastman Chemical Company, P.O. Box 511, Kingsport, TN, 37662-5075, US

(21) Appl. No.: 11/257,432

(22) Filed: Oct. 24, 2005

(51) Int. Cl. A61K 9/48; A61K 9/14

(52) U.S. Cl. 424/451; 424/488

(57) ABSTRACT

Pharmaceutical preparations and methods that contain enteric polymers formulated in liquid dosage forms. The preparations and methods provide enteric properties of delayed and then sustained release, without the need for expensive tableting or coating processes.

### FIELD OF THE INVENTION

This invention relates to the field of pharmaceutical preparations, and more specifically, to liquid dosage forms having enteric properties of delayed and then sustained release.

### BACKGROUND OF THE INVENTION

Enteric polymers exhibit a pH-dependent solubility in aqueous media and are commonly used for tablets and particle coatings in preparing oral dosage forms. Enteric polymers provide resistance to gastric fluids, but they are readily soluble in intestinal fluid. As such, enteric polymers prevent active drug ingredients in pharmaceutical preparations from disintegrating in the stomach while allowing the pharmaceutically active ingredient to be released once the dosage form has passed into the small intestinal tract. Thus polymeric materials suitable for enteric coatings are typically insoluble in a low pH medium having a value less than about 3.5, but soluble in a higher pH medium having a value greater than about 5.0.

Enteric protection is desirable when 1) the active substance is affected by the gastric acid in the stomach; 2) the active substance irritates the stomach; 3) there is a need to deliver the active to a particular site of the intestine; 4) there is a need to provide delayed release; or 5) taste masking is required. Enteric polymers currently used to coat pharmaceutical dosage forms include cellulose, vinyl and acrylic derivatives.

The art of using enteric polymers in pharmaceutical applications is known. See, for example, “Polymers For Enteric Coating Applications,” authored by G. Agyilirah and G. S. Banker, documented in chapter 3 of “Polymers For Controlled Drug Delivery” (P. J. Tarcha, ed., CRS Press, 1991); “Aqueous Polymeric Coatings for Pharmaceutical Dosage Forms”, 2nd edition, edited by J. McGinity (Marcel Dekker, NY, 1997), and especially the chapter “The chemistry and applications of cellulosic polymers for enteric coatings of solid dosage forms,” by Wu et al. See also, a paper written by Palmieri in Drug Development and Industrial Pharmacy (26(8), 837-845, 2000) entitled, “Polymers with pH dependent solubility:

Possibility of use in the formulation of gastroresistant and controlled-release matrix tablets”.

Pharmaceutical compositions intended for oral administration are typically solid dosage forms (e.g., tablets) or liquid preparations (e.g., solutions or suspensions). Liquid oral pharmaceutical compositions require a suitable solvent or carrier system to dissolve or disperse the active agent to enable the composition to be administered to a patient. As shown above, prior systems providing enteric protection have been directed to the delivery of active agents that are in dosage forms that are initially dry or in a solid state. There have only been a few systems that provide liquid formulations with enteric protection or those that would be retained in the stomach for a sustained period of time. Typically enteric protection for liquid preparations is provided by coating capsules with enteric polymers, see for example U.S. Pat. No. 6,635,281 and U.S. Pat. No.6,120,803.

JP 59193816 discloses a preparation of enteric soft capsules prepared by dissolving gelatin in an alkaline solution that contains membrane-forming substances, such as C-A-P. Banner also reported a clear softgel capsule made using rotary die encapsulation technology that has enteric protection incorporated in the shell of the capsule (Controlled Release Society annual meeting, 2004).

U.S. Pat. No. 4,727,109 discloses a pharmaceutical preparation having an active substance of low solubility in water and gastric juices that has an initial liquid carrier system. In this carrier system adjuvant substances are added to form a membrane around the liquid drops of the carrier system. This membrane formation results in delayed release of the active and produces a delayed-action drug effect. However, in this carrier system, hydrophobic components and a stabilizer are required.

There remains a need in the art for liquid dosage forms having enteric properties of delayed and/or sustained release, without the need for expensive tableting or coating processes.

### SUMMARY OF THE INVENTION

The present invention pertains to pharmaceutical preparations in liquid dosage forms encapsulated for oral administration comprising a mixture of: (1) a pharmaceutically active substance in which enteric protection is desired such as a non-steroid anti-inflammatory drug in an amount sufficient to provide a therapeutic effect when administered; (2) a cellulose acetate phthalate (C-A-P) at a concentration from about 5% to about 15% by weight based on the total weight of the preparation; (3) a solvent that includes polyethylene glycol at a molecular weight from about 200 to about 600 and/or propylene carbonate, wherein the concentration of polyethylene glycol is from about 50% to about 80% by weight based on the total weight of the preparation and the concentration of propylene carbonate is from 0% to about 15% by weight based on the total weight of the preparation; and (4) triacetin at a concentration from 0% to about 30% of the C-A-P weight.

The present invention also pertains to oral dosage forms, comprising a mixture of: a pharmaceutically active substance in an amount sufficient to provide a therapeutic effect when administered, a solvent comprising one or more of: acetone, ethyl acetate, ethyl alcohol, propylene glycol, polyethylene glycol with a molecular weight from about 200 to about 2000, or propylene carbonate at a concentration from about 20% to about 98% by weight based on the total weight of the dosage form, an enteric polymer comprising one or more of a cellulose polymer derivative, a vinyl polymer derivative, or an acrylic polymer derivative at a concentration from about 2% to about 80% by weight based on the total weight of the dosage form, and a plasticizer comprising one or more of a phthalate, a phosphate, a citrate, an adipate, a tartrate, a sebacate, a succinate, a glycolate, a glyceroalte, a benzoate, or a myristate at a concentration from 0% to about 30% of the enteric polymer weight, wherein upon administration about 5% or less of the active is released in a pH medium of 3.5 or less.

Another aspect of the present invention relates to a liquid dosage form, comprising a mixture of: a pharmaceutically active substance in which enteric protection is desired in an amount sufficient to provide a therapeutic effect when administered, an enteric polymer comprising one or more of a cellulose acetate phthalate (C-A-P), a cellulose acetate trimellitate (C-A-T), a cellulose acetate succinate (C-A-S), a hydroxypropyl methyl cellulose phthalate (HPMCP), a carboxymethyl ethylcellulose (CMEC), a hydroxypropyl methyl cellulose acetate succinate (HPMCAS), a polyvinyl acetate phthalate (PVAP), a copolymer of methacrylic acid and methyl methacrylate or ethyl acrylate, or a terpolymer of methacrylic acid, methacrylate, and ethyl acrylate, at a concentration from about 5% to about 50% by weight based on the total weight of the dosage form, and a solvent comprising one or more of propylene carbonate, polyethylene glycol with a molecular weight from about 200 to about 600, acetone, ethyl acetate, ethyl alcohol, or propylene glycol at a concentration from about 50% to about 95% by weight based on the total weight of the dosage form, wherein upon administration about 5% or less of the active is released in a pH medium of 3.5 or less and the active is then fully released in greater than about 2 hours or more in a pH medium of 5.0 or greater.

### BRIEF DESCRIPTION OF THE DRAWINGS

FIG. 1. shows the release profiles of ibuprofen from experimental runs #3 and #4.

FIG. 2. shows the release profile of ibuprofen from experimental runs #4 and #5.

FIG. 3. shows the release profile of ibuprofen from experimental runs #2 and #8.

### DETAILED DESCRIPTION OF THE INVENTION

The present invention may be understood more readily by reference to the following detailed description of preferred embodiments of the invention and the Examples included therein.

Before the present preparations and compositions are disclosed and described, it is to be understood that this invention is not limited to a specific method or to a particular formulation, as such may, of course, vary. It is also to be understood that the terminology used herein is for the purpose of describing particular embodiments only and is not intended to be limiting.

In this specification, the singular forms “a”, “an” and “the” include plural referents unless the context clearly dictates otherwise.

### Definitions

Listed below are definitions of various terms used to describe this invention. These definitions apply to the terms as they are used throughout this specification, unless otherwise indicated in specific instances.

As used herein, the term “mixture” means a combination of two or more substances resulting in a solution, dispersion or suspension.

As used herein, the term “solution” means a liquid preparation that contains one or more soluble active ingredients dissolved in a solvent.

As used herein, the term “dispersion” means a liquid preparation that contains finely divided, active ingredients dispersed in a solvent.

As used herein, the term “suspension” means a liquid preparation that contains finely divided, undissolved active ingredients suspended in a solvent.

As used herein, the term “pharmaceutical capsule” refers to any capsule that dissolves in water or gastric fluid including but not limited to hard or soft capsules in gelatin or non-gelatin varieties.

As used herein, the term “liquid dosage forms” refers to solutions, suspensions, or dispersions of the active or the active optionally in combination with pharmaceutically acceptable ingredients such as enteric polymers or solvents, in a liquid.

As used herein, the term “delayed and then sustained release” means with respect to the dosage forms of this invention that there is delayed release as the dosage remains in the stomach with very little active released and then sustained release as the active is slowly released after it enters the small intestine.

As used herein, the term “release profile” refers to the rate at which the active is released.

As used herein, the term “oral administration” refers to administration by capsule, liquid suspension or oral gavage, etc.

As used herein, the term “active” refers to an agent, drug, compound, or other substance, or composition and mixtures thereof that provide some pharmacologic, often beneficial, effect. Reference to a specific active shall include where appropriate the active and its pharmaceutically acceptable salts.

As used herein, the term “therapeutically effective amount” refers to the amount of the active agent needed to affect the desired pharmacologic, often beneficial, result.

The active ingredients are present in the dosage forms in therapeutically effective amounts; these amounts produce the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the active ingredient comprises about 0.1 to about 80 weight percent based on the total weight of the dosage form, for example the active may comprise about 2 to about 75 weight percent, or about 5 to 50 weight percent.

The dosage forms of the invention find use, for example, in humans or other animals. The environment of use is a fluid environment for purposes of this invention primarily includes the fluid environment of the stomach and the upper intestinal tract or small intestine. A single dosage form or several dosage forms can be administered to a subject during a therapeutic program.

A variety of enteric polymers may be used in the present invention. When used according to the invention, the polymers provide pharmaceutical preparations and methods that achieve some of the enteric-coating properties without the need for expensive coating or tableting processes. We have found that enteric polymers such as cellulose acetate phthalate (C-A-P) to be especially useful when formulated in a liquid dosage form to provide enteric-release properties to the formulations, as further described below.

The present invention thus provides pharmaceutical preparations in liquid dosage forms comprising a pharmaceutically active substance, a solvent, an enteric polymer and optionally a plasticizer.

The enteric polymers may be formulated in liquid dosage forms by a variety of methods, such as by: (i) combining therapeutically effective amounts of the active substances with solvents to obtain mixtures and (ii) incorporating enteric polymers into the mixtures. The liquid dosage forms may also be prepared according to the invention by dissolving the enteric polymers in the solvents to obtain mixtures, and then incorporating the active substances into the solvent mixtures. A plasticizer may then be added to the mixtures when the enteric polymers are dissolved. The viscosity of the resulting mixtures varies depending on the composition and method selected and may be as low as about 400 cp but may be as high as about 25,500 cp or even higher. The resulting mixtures are suitable for oral administration and encapsulation within pharmaceutical capsules. The resulting mixtures could also be administered orally as a suspension or by oral gavage.

Without wishing to be bound by any theory, in the liquid dosage forms of the present invention, the enteric polymers appear to function as phase changers. Upon contacting water or gastric fluid, the capsule dissolves and the enteric polymer in the filling mixture appears to congeal and form a solid mass with the active entrapped or retained therein, and as such, typically only a small amount of active would be expected to release in the stomach. For example as little as 5%, or even less, of the active would be released. When exposed thereafter to a higher pH, such as that in the intestinal tract, the enteric polymer starts to disintegrate layer by layer, gradually releasing the active substance. Typically, more than about 2 hours would be required for the active substance to be fully released. For example, the active may require up to about 7 hours or more to be fully released depending on the concentration of the components in the composition. Again, without being bound by any theory, delayed release and then sustained release action may be achieved.

Suitable enteric polymers include polymers that are typically insoluble in a low pH medium having a value less than about 3.5, but soluble in a higher pH medium having a value greater than about 5.0. For example, cellulose, vinyl and acrylic derivatives are suitable enteric polymers. Exemplary enteric polymers may include one or more of the following: cellulose acetate phthalates (C-A-P), cellulose acetate trimellitates (C-A-T), cellulose acetate succinates (C-A-S), hydroxypropyl methyl cellulose phthalates (HPMCP), carboxymethyl ethylcelluloses (CMEC), hydroxypropyl methyl cellulose acetate succinates (HPMCAS), polyvinyl acetate phthalates (PVAP), copolymers of methacrylic acid and methyl methacrylate or ethyl acrylate, or terpolymers of methacrylic acid, methacrylate, and ethyl acrylate, and the like. Especially suitable enteric polymers are the cellulose acetate phthalates (C-A-P).

In another aspect of the invention modifiers may be optionally added to the enteric polymers to alter the release profile of the active. U.S. Pat. No. 4,077,407 describes specific polymers and derivatives useful as enteric polymers and modifiers for enteric coatings and the relevant portions of which are incorporated herein by reference.

Exemplary modifiers include one or more of the following: cellulose acetates, cellulose diacetates, cellulose triacetates, cellulose propionates, cellulose acetate proprionates, and cellulose acetate butyrates, cellulose triacylates, cellulose trivalerates, cellulose trilaurates, cellulose tripalmitates, cellulose trisuccinates, cellulose triheptylates, cellulose tricaprylates, cellulose trioctanoates, cellulose tripropionates, polymeric cellulose esters and copolymeric cellulose esters such as mono, di, or tricellulose acylates, cellulose diesters such as cellulose diacylates, cellulose disuccinates, cellulose dipalmitates, cellulose dioctanoates, cellulose dicaprylates, or cellulose dipentanates, or esters prepared from acyl anhydrides or acyl acids such as cellulose acetate valerates, cellulose acetate succinates, cellulose propionate succinates, cellulose acetate octanoates, cellulose valerate palmitates, cellulose acetate palmitates, or cellulose acetate heptanoates.

The concentration of the enteric polymers or the enteric polymers with modifiers may vary within a wide range, for example from about 2% to about 80%, or from about 5% to about 50%, or from about 10% to about 30% by weight based on the total weight of the dosage form.

Any solvent or mixtures of solvents capable of dissolving the enteric polymers are suitable for use in the present invention. For example, acetone, ethyl acetate, ethyl alcohol, polyethylene glycols, propylene glycol, propylene carbonate, or mixtures thereof, are suitable solvents. Especially suitable solvents are polyethylene glycols, propylene carbonate, and mixtures thereof. Polyethylene glycols having, for example, an average molecular weight from about 200 to about 2000 are suitable. Especially suitable polyethylene glycols have an average molecular weight from about 200 to about 600. However, for polyethylene glycols that are not typically liquids at room temperature or with molecular weights greater than 600, a co-solvent such as acetone, ethyl acetate, ethyl alcohol, or polyethylene glycols with an average molecular weight from about 200 to about 600 may be used to pre-dissolve the higher molecular weight polyethylene glycols. The concentration of the solvents or solvent mixtures may vary within a wide range, for example from about 20% to about 98% by weight, or from about 50% to about 95%, or from about 50% to about 80% by weight based on the total weight of the dosage form. Suitable solvents also include mixtures of solvents, such as mixtures of polyethylene glycol 400 and propylene carbonate, for example, with polyethylene glycol 400 at a concentration, for example, of about 50% to about 80% by weight, with propylene carbonate at a concentration from 0% up to about 15% by weight.

A wide variety of pharmaceutical actives may be used in the present invention. Typically, any pharmaceutically active substance in which enteric protection is desired would be suitable. As such, any one or more of the following are suitable pharmaceutical actives: analgesics, anti-inflammatory agents, anti-helminthics, anti-arrhythmic agents, anti-bacterial agents, anti-viral agents, anti-coagulants, anti-depressants, anti-diabetics, anti-epileptics, anti-cancer agents, anti-fungal agents, anti-gout agents, anti-hypertensive agents, anti-malarials, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, lipid regulating agents, anti-anginal agents, cox-2 inhibitors, antioxidant agent, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, hormones, steroids, steroid antagonists, vitamins, nutritional supplements, essential fatty acids or non-essential fatty acids. The pharmaceutical actives may be used in any amount sufficient to provide a therapeutic effect when administered. As such, the concentration may vary from as little as about 0.1%, by weight based on the total weight of the dosage form, or from about 1% up to about 50%, or up to even 75%, or more, depending on the active selected.

In another aspect of the present invention, a plasticizer may be used. Without being bound by any theory, the plasticizer may increase the flexibility of the enteric polymer during the phase change to a solid mass. As the solid mass is formed, the plasticizer may prevent the formation of cracks or other defects within the solid mass. As such, the active may be more uniformly entrapped within the resulting solid thereby improving the release profile of the active.

The plasticizer may be added to the solvent mixture after both the active substance and the enteric polymer are dissolved therein. Suitable plasticizers include phthalates, phosphates, citrates, adipates, tartrates, sebacates, succinates, glycolates, glyceroaltes, benzoates, myristates, and mixtures thereof. U.S. Pat. No. 4,077,407 describes specific plasticizers suitable for enteric coatings and the relevant portions of which are incorporated herein by reference.

Exemplary plasticizers include dialkyl phthalates; dicycloalkyl phthalates; diaryl phthalates and mixed alkyl-aryl phthalates as represented by dimethyl phthalates, dipropyl phthalates, di(2-ethylhexyl)-phthalates, di-isopropyl phthalates, diamyl phthalates and dicapryl phthalates; alkyl and aryl phosphates such as tributyl phosphates, trioctyl phosphates, tricresyl phosphates, trioctyl phosphates, tricresyl phosphates and triphenyl phosphates; alkyl citrates and citrate esters such as tributyl citrates, triethyl citrates, and acetyl triethyl citrates; alkyl adipates such as dioctyl adipates, diethyl adipates and di(2-methoxyethyl)-adipates; dialkyl tartrates such as diethyl tartrates and dibutyl tartrates; alkyl sebacates such as diethyl sebacates, dipropyl sebacates and dinonyl sebacates; alkyl succinates such as diethyl succinates and dibutyl succinates; alkyl glycolates, alkyl glycerolates, glycol esters and glycerol esters such as glycerol diacetates, glycerol triacetates, glycerol monolactate diacetates, methyl phythayl ethyl glycolates, butyl phthalyl butyl glycolates, ethylene glycol diacetates, ethylene glycol dibutyrates, triethylene glycol diacetates, triethylene glycol dibutyrates, or triethylene glycol dipropionates. For example, one or more of diethyl phthalate, triacetin, acetylated monoglycerides, butyl phthalyl butyl glycolate, tributyl citrate or triethyl citrate could be used. We have found triacetin to be especially suitable. The amount of plasticizer that is used will vary based on the enteric polymer chosen. Generally, the concentration of the plasticizer is from 0% up to about 30% of the enteric polymer weight.

The invention will be more readily understood by reference to the following examples. There are, of course, many other forms of this invention that will become obvious to one skilled in the art, once the invention has been fully disclosed, and it will accordingly be recognized that these examples are given for the purpose of illustration only, and are not to be construed as limiting the scope of this invention in any way.

### EXAMPLES

In the following examples, ibuprofen (DASTECH International, Harrison, N.J.) was chosen as the model active because it is a non-steroidal anti-inflammatory drug (NSAID). The formulations used in these studies also contained C-A-P NF pellets (Eastman Chemical Company, Kingsport, Tenn.), triacetin (Eastman Chemical Company, Kingsport, Tenn.), PEG 400 (Sigma-Aldrich, St Louis, Mo.), and propylene carbonate (Sigma-Aldrich).

Preparation of the Capsule Filling Mixtures Used Below

In an 8 oz glass jar, the stated amounts of PEG 400 and propylene carbonate were weighed, and the desired amount of ibuprofen was then added to the solvents gradually while stirring at room temperature. After the ibuprofen went into solution, the stated amount of C-A-P was gradually added to the solvent mixture. When the C-A-P dissolved, triacetin was then added to the mixture. A relatively clear, homogeneous mixture was obtained. The viscosity of the mixture was in the range of about 400 to about 25,500 cp. The mixture was then held at room temperature to allow air bubbles to degas from the mixture. After degassing, the resulting mixture would be suitable for encapsulation within a pharmaceutical capsule.

Preparation of the Soft Capsules Used Below

Air filled oval hex twist soft capsules (Banner Pharmacaps, Chatsworth, Calif.) were used in these studies. When the tab of a capsule was twisted off, a small opening was obtained. A 5 ml syringe (Becton, Dickson and Company, Franklin Lakes, N.J.) with a 21 gauge needle (Becton, Dickson and Company) was used to fill the above prepared mixture into the capsule. A seal for the opening of the filled capsule was prepared by melting the twisted off tab in water and then adjusting the Viscosity of the mixture with water and heat.

Dissolution Test:

Dissolution apparatus (dissolution system 2100A, DISTEK, North Brunswick, N.J.) and a USP test method using apparatus II was used to determine the release profile of ibuprofen in pH 1.2 and 6.8 buffer. The buffer solutions were prepared according to the methods described in USP 25/NF 20 (USP, 2002). The pH 1.2 solution was 0.1 N hydrochloric acid (Merck KGaA, Darmstadt, Germany); the pH 6.8 buffer was made by using 3 to 1 ratio of 0.1 N hydrochloric acid to 0.2 N tribasic sodium phosphate (Sigma-Aldrich, Steinheim, Germany). The pH value of the 6.8 buffer was adjusted by using 2.0 N sodium hydroxide solution (VWR, West Chester, Pa.) as needed. For each formulation, six soft capsules were prepared according to the above described procedures.

The dissolution tests were conducted at 37° C. and the peddle rate was set at 50 rpm. The capsules were tested in 750 ml of pH 1.2 acid solution for two hours. The testing medium was then changed to pH 6.8 by adding 250 ml of 0.2 N tribasic sodium phosphate solution. The tested capsules were maintained in the testing vessels during the pH change of the testing medium, and tested in the high pH medium for another two to seven hours depending on the formulation. The dissolution test was terminated when all C-A-P dissolved and the trapped active went into the solution. One ml samples were drawn over time and the concentrations of the ibuprofen were determined by a HPLC method.

We found that in a solution having a pH of 1.2, from 1.8% -7.0% of the ibuprofen was released. In a pH 6.8 buffer, the ibuprofen was fully released within 2-7 hours, depending on the formulation. In general, we found that the more C-A-P in the formulation, the slower the ibuprofen was released.

### Example 1

30.00 g of ibuprofen was dissolved in the mixture of 132.01 g of PEG 400 and 15.01 g of propylene carbonate under stirring at room temperature. After the ibuprofen dissolved, 20.00 g of C-A-P was gradually added to the mixture. When the C-A-P dissolved, 3.00 g of triacetin was added. The filling mixture was allowed to degas prior to being encapsulated into a pharmaceutical capsule.

### Example 2

A series of formulations were prepared. Table 1 lists the formulations of the experiments that were conducted.

TABLE 1
Experiments Performed.

| | | Propylene | C-A-P | Triacetin | Ibuprofen |
|---|---|---|---|---|---|
| Run | PEG 400 (g) | Carbonate (g) | (g) | (g) | (g) |
| 1 | 132.01 | 15.01 | 20.00 | 3.00 | 30.00 |
| 2 | 157.02 | 0.00 | 10.01 | 3.02 | 30.01 |
| 3 | 130.01 | 30.02 | 10.00 | 0.00 | 30.00 |
| 4 | 110.01 | 30.00 | 30.01 | 0.00 | 30.00 |
| 5 | 101.01 | 30.02 | 30.00 | 9.02 | 30.01 |
| 6 | 132.00 | 15.00 | 20.00 | 3.01 | 30.01 |
| 7 | 140.00 | 0.00 | 30.01 | 0.00 | 30.00 |
| 8 | 127.01 | 30.01 | 10.00 | 3.00 | 30.01 |
| 9 | 160.00 | 0.00 | 10.02 | 0.00 | 30.01 |
| 10 | 131.01 | 0.00 | 30.01 | 9.02 | 30.00 |
| 11 | 132.00 | 15.01 | 20.00 | 3.01 | 30.00 |

### Example 3

The viscosity of the filling mixtures was measured for each of the formulations prepared above (as listed in Table 1) using a Brookfield Viscometer (model DV-I+). The viscosity data is shown in Table 2.

TABLE 2
Viscosity Data of the Filling Mixtures.

| | | | | | | Viscosity |
|---|---|---|---|---|---|---|
| Run | Spindle | Speed/RPM | Temp. | Volume | Wt./g | (cp) |
| 1 | 27 | 6.0 | 37° C. | 10.5 | 11.57 | 3,609 |
| 2 | 27 | 30.0 | 37° C. | 10.5 | 11.7 | 685.9 |
| 3 | 27 | 30.0 | 37° C. | 10.5 | 12.07 | 457.0 |
| 4 | 27 | 0.6 | 37° C. | 10.5 | 12.05 | 15,700 |
| 5 | 27 | 1.5 | 37° C. | 10.5 | 11.87 | 12,230 |
| 6 | 27 | 6.0 | 37° C. | 10.5 | 12.05 | 3,487 |
| 7 | 27 | 0.6 | 37° C. | 10.5 | 11.95 | 25,390 |
| 8 | 27 | 0.6 | 37° C. | 10.5 | 11.7 | 425.8 |
| 9 | 27 | 30.0 | 37° C. | 10.5 | 11.48 | 695.3 |
| 10 | 27 | 0.6 | 37° C. | 10.5 | 12.26 | 22,110 |
| 11 | 27 | 6.0 | 37° C. | 10.5 | 11.78 | 3,136 |

### Example 4

Dissolution tests were performed for eleven formulations. The tests were conducted in a pH 1.2 acid solution for two hours and then in a pH 6.8 buffer solution for an additional two to seven hours depending on the formulation. The experiments were terminated when all of the solid C-A-P appeared to dissolve and the entrapped active was released. Table 3 lists the results of the dissolution test.

TABLE 3
Dissolution Results.

| | | | Propylene | | |
|---|---|---|---|---|---|
| Run | C-A-P | Triacetin | carbonate | Time | % Ibuprofen |
| # | (g) | (g) | (g) | (minutes) | released |
| 1 | 20.00 | 3.00 | 15.01 | 10.0 | 0.0 |
| | | | | 30.0 | 0.4 |
| | | | | 60.0 | 1.4 |
| | | | | 90.0 | 2.6 |
| | | | | 120.0 | 3.6 |
| | | | | 127.0 | 13.4 |
| | | | | 136.0 | 24.4 |
| | | | | 150.0 | 41.6 |
| | | | | 165.0 | 58.4 |
| | | | | 180.0 | 75.3 |
| | | | | 210.0 | 94.6 |
| | | | | 270.0 | 100.0 |
| | | | | 300.0 | 98.6 |
| | | | | 360.0 | 102.2 |
| | | | | 420.0 | 100.0 |
| 2 | 10.01 | 3.02 | 0.00 | 10.0 | 0.1 |
| | | | | 30.0 | 1.6 |
| | | | | 60.0 | 3.6 |
| | | | | 90.0 | 3.9 |
| | | | | 120.0 | 5.6 |
| | | | | 125.0 | 22.4 |
| | | | | 135.0 | 40.1 |
| | | | | 150.0 | 65.3 |
| | | | | 165.0 | 83.0 |
| | | | | 180.0 | 92.8 |
| | | | | 210.0 | 98.6 |
| | | | | 270.0 | 103.0 |
| | | | | 300.0 | 100.0 |
| 3 | 10.00 | 0.00 | 30.02 | 10.0 | 0.2 |
| | | | | 30.0 | 2.4 |
| | | | | 60.0 | 4.0 |
| | | | | 90.0 | 4.1 |
| | | | | 120.0 | 4.4 |
| | | | | 125.0 | 17.5 |
| | | | | 135.0 | 35.9 |
| | | | | 150.0 | 59.8 |
| | | | | 165.0 | 72.6 |
| | | | | 180.0 | 93.3 |
| | | | | 210.0 | 98.1 |
| | | | | 270.0 | 99.4 |
| | | | | 300.0 | 100.0 |
| 4 | 30.01 | 0.00 | 30.00 | 10.0 | 0.0 |
| | | | | 30.0 | 0.6 |
| | | | | 60.0 | 1.2 |
| | | | | 90.0 | 1.7 |
| | | | | 120.0 | 2.0 |
| | | | | 125.0 | 9.6 |
| | | | | 135.0 | 20.1 |
| | | | | 150.0 | 36.0 |
| | | | | 165.0 | 49.5 |
| | | | | 180.0 | 62.4 |
| | | | | 210.0 | 78.6 |
| | | | | 270.0 | 94.0 |
| | | | | 330.0 | 100.1 |
| | | | | 360.0 | 100.0 |
| 5 | 30.00 | 9.02 | 30.02 | 10.0 | 0.0 |
| | | | | 30.0 | 0.7 |
| | | | | 60.0 | 1.4 |
| | | | | 90.0 | 2.0 |
| | | | | 120.0 | 2.7 |
| | | | | 125.0 | 9.7 |
| | | | | 135.0 | 21.5 |
| | | | | 150.0 | 38.9 |
| | | | | 165.0 | 56.2 |
| | | | | 180.0 | 66.8 |
| | | | | 210.0 | 81.3 |
| | | | | 270.0 | 98.9 |
| | | | | 310.0 | 99.2 |
| | | | | 340.0 | 100.0 |
| 6 | 20.00 | 3.01 | 15.00 | 10.0 | 0.0 |
| | | | | 30.0 | 1.0 |
| | | | | 60.0 | 2.1 |
| | | | | 90.0 | 2.7 |
| | | | | 120.0 | 4.9 |
| | | | | 125.0 | 12.6 |
| | | | | 135.0 | 24.3 |
| | | | | 150.0 | 40.7 |
| | | | | 165.0 | 60.5 |
| | | | | 180.0 | 76.8 |
| | | | | 210.0 | 94.7 |
| | | | | 270.0 | 100.0 |
| 7 | 30.01 | 0.00 | 0.00 | 10.0 | 0.0 |
| | | | | 40.0 | 0.7 |
| | | | | 80.0 | 1.6 |
| | | | | 120.0 | 1.8 |
| | | | | 125.0 | 7.2 |
| | | | | 135.0 | 11.5 |
| | | | | 150.0 | 20.4 |
| | | | | 165.0 | 28.0 |
| | | | | 180.0 | 34.1 |
| | | | | 210.0 | 45.5 |
| | | | | 270.0 | 63.2 |
| | | | | 360.0 | 84.1 |
| | | | | 504.0 | 100.0 |
| 8 | 10.00 | 3.00 | 30.01 | 10.0 | 0.4 |
| | | | | 30.0 | 2.2 |
| | | | | 60.0 | 3.7 |
| | | | | 90.0 | 4.8 |
| | | | | 120.0 | 4.7 |
| | | | | 125.0 | 16.1 |
| | | | | 135.0 | 28.7 |
| | | | | 150.0 | 48.4 |
| | | | | 165.0 | 65.9 |
| | | | | 180.0 | 78.4 |
| | | | | 210.0 | 91.9 |
| | | | | 270.0 | 98.2 |
| | | | | 330.0 | 100.0 |
| 9 | 10.02 | 0.00 | 0.00 | 10.0 | 0.1 |
| | | | | 30.0 | 1.8 |
| | | | | 60.0 | 7.9 |
| | | | | 90.0 | 6.8 |
| | | | | 120.0 | 7.1 |
| | | | | 125.0 | 21.8 |
| | | | | 135.0 | 37.2 |
| | | | | 150.0 | 58.3 |
| | | | | 165.0 | 75.0 |
| | | | | 180.0 | 86.4 |
| | | | | 210.0 | 96.7 |
| | | | | 270.0 | 99.7 |
| | | | | 330.0 | 100.0 |
| 10 | 30.01 | 9.02 | 0.00 | 10.0 | 0.0 |
| | | | | 40.0 | 0.8 |
| | | | | 80.0 | 1.4 |
| | | | | 120.0 | 1.8 |
| | | | | 125.0 | 4.9 |
| | | | | 135.0 | 8.7 |
| | | | | 150.0 | 15.6 |
| | | | | 165.0 | 22.2 |
| | | | | 180.0 | 28.3 |
| | | | | 210.0 | 38.2 |
| | | | | 270.0 | 54.3 |
| | | | | 330.0 | 67.4 |
| | | | | 450.0 | 86.6 |
| | | | | 570.0 | 97.7 |
| | | | | 660.0 | 100.0 |
| 11 | 20.00 | 3.01 | 15.01 | 10.0 | 0.0 |
| | | | | 40.0 | 1.2 |
| | | | | 80.0 | 2.5 |
| | | | | 120.0 | 3.5 |
| | | | | 125.0 | 11.1 |
| | | | | 135.0 | 19.4 |
| | | | | 150.0 | 34.0 |
| | | | | 165.0 | 49.9 |
| | | | | 180.0 | 65.4 |
| | | | | 210.0 | 87.4 |
| | | | | 270.0 | 100.1 |
| | | | | 330.0 | 99.7 |
| | | | | 350.0 | 100.0 |

### Example 5

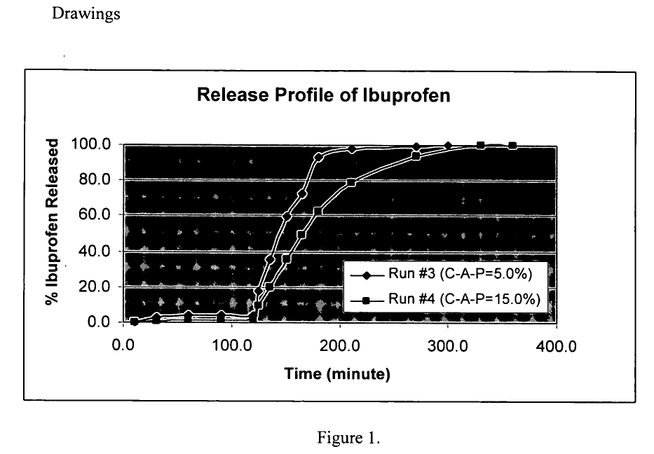

The following examples establish that the concentration of the components in the formulations influence the release profile of the active substances. For example, FIG. 1 displays the release profile of ibuprofen from experimental runs #3 and #4. The formulations for both runs had the same concentration of triacetin at 0.0% and propylene carbonate at 15.0%, but the concentration of the C-A-P was varied from 5.0-15.0%. As illustrated in FIG. 1, the more C-A-P in the formulation, the slower the release rate.

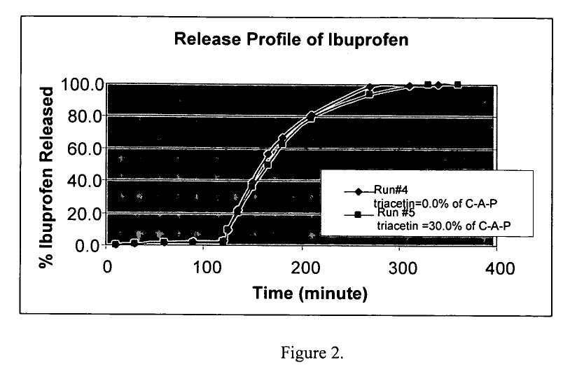

FIG. 2 shows the release profile of the active substance from experimental runs #4 and #5. These formulations consisted of C-A-P at 15.0%, propylene carbonate at 15.0%, and triacetin ranging from 0.0-30.0% of C-A-P weight. As illustrated in FIG. 2, the triacetin does not appear to have a significant effect on the release profile of ibuprofen in these formulations; however, the triacetin does appear to have an impact on the characteristics of the solid mass formed. Therefore, in other formulations with different actives, solvents, enteric polymers and plasticizers a more significant impact on the release profile may be observed.

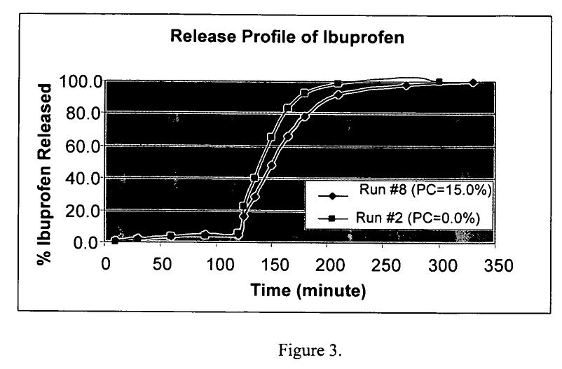

FIG. 3 shows the release profiles of ibuprofen from experimental runs #2 and #8. The C-A-P concentration was maintained at 5.0% and the triacetin was held at 30.0% of C-A-P weight in both runs. The concentration of propylene carbonate was varied from 0.0-15.0%. As illustrated in FIG. 3, the concentration of propylene carbonate also affected the release profile of the ibuprofen. The more propylene carbonate in the formulation, the slower the release rate.

### Example 6

The following studies were conducted to determine the stability of the filling mixtures. Stability of the mixtures was determined by analyzing the hydrolysis rate of C-A-P in the formulation as indicated by an increase in the amount of phthalic acid. The mixtures were kept in a glass jar in an oven at 40° C. and 75% relative humidity for three months. These conditions were designed to stimulate two years of shelf life in an ambient environment. The specification for free phthalic acid in the commercial C-A-P is less than 3.0%. The hydrolysis data is shown in table 4.

TABLE 4
Hydrolysis Data of C-A-P.

| Sample I.D. | % phthalic acid before oven | % phthalic acid after oven |
|---|---|---|
| 1 | 2.0 | 3.3 |
| 2 | 1.6 | 3.0 |
| 3 | 2.2 | 4.0 |
| 4 | 2.2 | 3.4 |
| 5 | 2.7 | 4.0 |
| 6 | 2.4 | 3.6 |
| 7 | 2.5 | 4.1 |
| 8 | 2.6 | 4.4 |
| 9 | 2.4 | 4.2 |
| 10 | 2.6 | 3.5 |
| 11 | 2.4 | 4.1 |

It should be noted that the formulation samples were kept at room temperature for about 6 months before the oven stability tests were performed. These tests were conducted to determine how the release profile of the ibuprofen changes with C-A-P hydrolysis. Based on the three month oven stability test, the maximum amount of phthalic acid in the hydrolyzed C-A-P samples was 4.4%.

Table 5 lists the release profile of ibuprofen at 4.4% hydrolysis of C-A-P with a formulation consisting of 15.0% of C-A-P and 15.0% of propylene carbonate.

TABLE 5
Release Profile of Ibuprofen at 4.4% Hydrolysis of C-A-P

| Time | % Ibuprofen released | % Ibuprofen released | |
|---|---|---|---|
| (minute) | (before oven test) | (after oven test) | % change |
| 120 | 0.0 | 0.0 | 0.0 |
| 130 | 16.2 | 16.5 | 2.0 |
| 140 | 29.8 | 30.3 | 1.8 |
| 150 | 41.2 | 41.8 | 1.6 |
| 160 | 50.7 | 51.4 | 1.5 |
| 170 | 58.7 | 59.5 | 1.3 |
| 180 | 65.4 | 66.2 | 1.2 |
| 190 | 71.0 | 71.8 | 1.1 |
| 200 | 75.7 | 76.4 | 1.0 |
| 210 | 79.6 | 80.3 | 0.9 |
| 220 | 82.9 | 83.6 | 0.8 |
| 230 | 85.7 | 86.3 | 0.7 |
| 240 | 88.0 | 88.6 | 0.6 |
| 250 | 90.0 | 90.4 | 0.5 |
| 260 | 91.6 | 92.0 | 0.5 |
| 270 | 93.0 | 93.3 | 0.4 |
| 280 | 94.1 | 94.4 | 0.4 |
| 290 | 95.1 | 95.4 | 0.3 |
| 300 | 95.9 | 96.1 | 0.3 |
| 310 | 96.5 | 96.8 | 0.3 |
| 320 | 97.1 | 97.3 | 0.2 |
| 330 | 97.6 | 97.7 | 0.2 |
| 340 | 98.0 | 98.1 | 0.2 |
| 350 | 98.3 | 98.4 | 0.1 |
| 360 | 98.6 | 98.7 | 0.1 |
| 370 | 98.8 | 98.9 | 0.1 |
| 380 | 99.0 | 99.1 | 0.1 |
| 390 | 99.2 | 99.2 | 0.1 |
| 400 | 99.3 | 99.4 | 0.1 |
| 410 | 99.4 | 99.5 | 0.1 |
| 420 | 99.5 | 99.6 | 0.1 |

The above data shows that the change in the release rate of ibuprofen was in the range of 0.0-2.0%. Therefore, it was concluded that all of the formulations were basically stable under the specified testing conditions.

In the drawings and specification, there have been disclosed typical preferred embodiments of the invention and, although specific terms are employed, they are used in a generic and descriptive sense only and not for purposes of limitation, the scope of the invention being set forth in the following claims. The invention has been described in detail with particular reference to preferred embodiments thereof, but it will be understood that variations and modifications can be effected within the spirit and scope of the invention.

1. A pharmaceutical preparation in liquid dosage form encapsulated for oral administration comprising a mixture of:
(1) a pharmaceutically active substance in which enteric protection is desired such as a non-steroid anti-inflammatory drug in an amount sufficient to provide a therapeutic effect when administered;
(2) a cellulose acetate phthalate (C-A-P) at a concentration from about 5% to about 15% by weight based on the total weight of the preparation;
(3) a solvent that includes polyethylene glycol at a molecular weight from about 200 to about 600 and/or propylene carbonate, wherein the concentration of polyethylene glycol is from about 50% to about 80% by weight based on the total weight of the dosage form and the concentration of propylene carbonate is from 0% to about 15% by weight based on the total weight of the dosage form; and
(4) triacetin at a concentration from 0% to about 30% of the C-A-P weight.

2. The pharmaceutical preparation of claim 1 wherein upon administration the active undergoes delayed release such that about 5% or less of the active is released in a pH medium of 3.5 or less and then sustained release such that the active is fully released in greater than about 2 hours or more in a pH medium of 5.0 or greater.

3. The pharmaceutical preparation of claim 1 wherein the polyethylene glycol has an average molecular weight of about 400.

4. The pharmaceutical preparation of claim 1 wherein the resulting mixture has a viscosity ranging from about 400 cp to about 25,500 cp.

5. A method of preparing the pharmaceutical preparation of claim 1 comprising combining the active substance with the solvent to obtain a mixture, incorporating the C-A-P into the solvent mixture and adding the triacetin to the solvent mixture when the C-A-P is dissolved.

6. A method of preparing the pharmaceutical preparation of claim 1 comprising combining the C-A-P with the solvent to obtain a mixture, incorporating the active substance into the solvent mixture and adding the triacetin to the solvent mixture when the active substance is dissolved.

7. An oral dosage form, comprising a mixture of:
a pharmaceutically active substance in an amount sufficient to provide a therapeutic effect when administered,
a solvent comprising one or more of: acetone, ethyl acetate, ethyl alcohol, propylene glycol, polyethylene glycol with a molecular weight from about 200 to about 2000, or propylene carbonate at a concentration from about 20% to about 98% by weight based on the total weight of the dosage form,
an enteric polymer comprising one or more of a cellulose polymer derivative, a vinyl polymer derivative, or an acrylic polymer derivative at a concentration from about 2% to about 80% by weight based on the total weight of the dosage form, and
a plasticizer comprising one or more of a phthalate, a phosphate, a citrate, an adipate, a tartrate, a sebacate, a succinate, a glycolate, a glyceroalte, a benzoate, or a myristate at a concentration from 0% to about 30% of the enteric polymer weight,
wherein upon administration about 5% or less of the active is released in a pH medium of 3.5 or less.

8. The oral dosage form of claim 7 wherein the resulting composition is a liquid suitable for oral administration and encapsulation within a pharmaceutical capsule.

9. The oral dosage form of claim 7 wherein upon administration the active is fully released in greater than about 2 hours or more in a pH medium of 5.0 or greater.

10. The oral dosage form of claim 7 wherein the release profile of the active can be modified by adjusting the concentrations of the components in the composition.

11. The oral dosage form of claim 7 wherein the pharmaceutically active substance is one or more of: analgesics, anti-inflammatory agents, anti-helminthics, anti-arrhythmic agents, anti-bacterial agents, anti-viral agents, anti-coagulants, anti-depressants, anti-diabetics, anti-epileptics, anti-cancer agents, anti-fungal agents, anti-gout agents, anti-hypertensive agents, anti-malarials, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, lipid regulating agents, anti-anginal agents, cox-2 inhibitors, antioxidant agents, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, hormones, steroids, steroid antagonists, vitamins, nutritional supplements, essential fatty acids, or non-essential fatty acids.

12. The oral dosage form of claim 7 wherein the polyethylene glycol has a molecular weight of about 200 to about 600.

13. The oral dosage form of claim 7 wherein the polyethylene glycol comprises polyethylene glycol 400.

14. The oral dosage form of claim 7 wherein the solvent system is one or more of polyethylene glycol with a molecular weight from about 200 to about 600 or propylene carbonate.

15. The oral dosage form of claim 14 wherein the solvent system comprises polyethylene glycol at a concentration from about 50% to about 80% by weight and propylene carbonate at a concentration from 0% to about 15% by weight.

16. The oral dosage form of claim 14 wherein the higher the concentration of the propylene carbonate in the formulation, the slower the rate of release of the active.

17. The oral dosage form of claim 7 wherein the enteric polymer is one or more of a cellulose acetate phthalate (C-A-P), a cellulose acetate trimellitate (C-A-T), a cellulose acetate succinate (C-A-S), a hydroxypropyl methyl cellulose phthalate (HPMCP), a carboxymethyl ethylcellulose (CMEC), a hydroxypropyl methyl cellulose acetate succinate (HPMCAS), a polyvinyl acetate phthalate (PVAP), a copolymer of methacrylic acid and methyl methacrylate or ethyl acrylate, or a terpolymer of methacrylic acid, methacrylate, and ethyl acrylate.

18. The oral dosage form of claim 7 wherein the enteric polymer is a cellulose acetate phthalate (C-A-P).

19. The oral dosage form of claim 7 wherein the concentration of the enteric polymer is from about 5% to about 50% by weight.

20. The oral dosage form of claim 7 wherein the concentration of the enteric polymer is from about 10% to about 30% by weight.

21. The oral dosage form of claim 7 wherein the higher the concentration of the enteric polymer in the formulation, the slower the rate of release of the active.

22. The oral dosage form of claim 7 wherein a modifier is optionally added to the enteric polymer.

23. The oral dosage form of claim 7 wherein the plasticizer is one or more of diethyl phthalate, triacetin, acetylated monoglycerides, butyl phthalyl butyl glycolate, tributyl citrate, or triethyl citrate.

24. The oral dosage form of claim 7 wherein the plasticizer is triacetin.

25. The oral dosage form of claim 7 wherein the resulting mixture has a viscosity ranging from about 400 to about 25,500 cp.

26. A method of preparing the oral dosage form of claim 7 comprising combining the active substance with the solvent system to obtain a mixture, incorporating the enteric polymer into the mixture and optionally adding the plasticizer to the mixture when the enteric polymer is dissolved.

27. A liquid dosage form, comprising a mixture of:
a pharmaceutically active substance in which enteric protection is desired in an amount sufficient to provide a therapeutic effect when administered,
an enteric polymer comprising one or more of a cellulose acetate phthalate (C-A-P), a cellulose acetate trimellitate (C-A-T), a cellulose acetate succinate (C-A-S), a hydroxypropyl methyl cellulose phthalate (HPMCP), a carboxymethyl ethylcellulose (CMEC), a hydroxypropyl methyl cellulose acetate succinate (HPMCAS), a polyvinyl acetate phthalate (PVAP), a copolymer of methacrylic acid and methyl methacrylate or ethyl acrylate, or a terpolymer of methacrylic acid, methacrylate, and ethyl acrylate, at a concentration from about 5% to about 50% by weight based on the total weight of the dosage form; and
a solvent comprising one or more of propylene carbonate, polyethylene glycol with a molecular weight from about 200 to about 600, acetone, ethyl acetate, ethyl alcohol, or propylene glycol at a concentration from about 50% to about 95% by weight based on the total weight of the dosage form,
wherein upon administration about 5% or less of the active is released in a pH medium of 3.5 or less and the active is then fully released in greater than about 2 hours or more in a pH medium of 5.0 or greater.

28. The pharmaceutical preparation of claim 7 wherein a plasticizer comprising one or more of diethyl phthalate, triacetin, acetylated monoglycerides, butyl phthalyl butyl glycolate, tributyl citrate, or triethyl citrate is added to the mixture at a concentration from 0% to about 30% of the enteric polymer weight.
